(19) United States
(12) Patent Application Publication
(10) Pub. No.: US 2013/0110087 A1
(43) Pub. Date: May 2, 2013

(54) System and Method for Clearing Medical Tubing

(71) Applicant: Jeffrey F. Kane, Worcester, MA (US)

(72) Inventor: Jeffrey F. Kane, Worcester, MA (US)

(21) Appl. No.: 13/578,932

(22) PCT Filed: Feb. 17, 2011
(86) PCT No.: PCT/US2011/025260

§ 371 Date: Jan. 3, 2013

Related U.S. Application Data
(60) Provisional application No. 61/305,291, filed on Feb. 17, 2010.

(51) Int. Cl. B08B 9/027; A61M 27/00

(52) U.S. Cl. CPC B08B 9/027; A61M 27/00
USPC 604/543; 15/104.03

(57) ABSTRACT

An apparatus for clearing fluid from medical tubing includes a first arm having a first friction reducing member, a second arm having a second friction reducing member, and a hinge connecting the first and second arms to form a body. The body has a generally longitudinal axis, and the first arm may be movable relative to the second arm. The first and second friction reducing members may be normally adjacent to and spaced from each other. The hinge may form an opening sized and shaped to receive the medical tubing. The opening, first friction reducing member, and second friction reducing member may be aligned generally along the longitudinal axis.

### PRIORITY

This patent application claims priority from U.S. Provisional Patent Application No. 61/305,291, filed Feb. 17, 2010, entitled, “System and Method for Clearing Medical Tubing,” and naming Jeffrey F. Kane as inventor, the disclosure of which is incorporated herein, in its entirety, by reference.

### TECHNICAL FIELD

This invention relates to wound drainage sets and, more particularly, to systems and methods for clearing unwanted residual fluids and occlusions from medical drain tubing.

### BACKGROUND ART

As is known in the art, wound drainage sets are typically administered following any number of surgical procedures to aid in the capture of excess bodily fluids during patient recovery. A typical wound drain has (1) an inlet having a flexible chamber with perforated holes, (2) a flexible silicone tubing attached to the inlet, and (3) a flexible silicone bulb to provide a reservoir for collecting the fluids. The inlet is placed subcutaneously in the wound site. The tube is typically about three feet in length and extends from the inlet (through an incision), and attaches to the reservoir/bulb located outside of the patient's body. Drainage systems of this type are commonly used post-operatively for up to two weeks (sometimes longer depending on the individual and type of surgical procedure).

In order to ensure adequate draining, the bulb and tubing must be emptied several times during patient recovery to clear possible occlusions. Emptying the bulb is rather straight forward and may be accomplished while the bulb is attached to the tubing. For example, a user may simply open a drain port located on the reservoir, and pour the excess fluid into a waste container for proper disposal. However, emptying or clearing the tubing is much more difficult and requires skill, knowledge and physical dexterity. In practice, clearing the tubing is referred to as “milking” or “stripping.” This process is accomplished by pinching the tubing closed with the thumb and index finger of one hand very close to the incision (e.g., where the tubing exits the body). This hand should remain stationary while keeping the tubing pinched so as to prevent the tubing from being pulled from the incision (which would cause excessive pain and discomfort to the patient). Using the other hand, the user must squeeze the tubing immediately adjacent the stationary hand, and draw their hand toward the drainage bulb, allowing the tubing to slide through the thumb and finger. This will cause any residual fluid and possible occlusions present in the tubing to be drawn, or stripped, toward and into the drainage bulb.

In many cases, patients require assistance from another individual or medical practitioner to perform the stripping process because of the difficulty in properly performing the procedure. Due to the force required to adequately pinch the tube and subsequently draw fluid toward the drainage reservoir with one hand, a greater force must be maintained on the hand closest to the incision to prevent the tubing from being pulled at the incision point. It is important to note that a great deal of friction is created when drawing the tubing through the thumb and index finger while simultaneously pinching the tubing closed. Much force is required to overcome this friction to produce the intended “squeegee” effect and to “milk or “strip” the tubing of any residual unwanted fluids.

### SUMMARY OF THE EMBODIMENTS

In a first embodiment of the present invention there is provided an apparatus for clearing fluid from a flexible tube (e.g., medical tubing). The apparatus includes a first arm having a first friction reducing member, and second arm having a second friction reducing member, and a hinge. The hinge (e.g., a living hinge) may connect the first and second arms to form a body that a has a generally longitudinal axis. The first arm may be moveable relative to the second arm, and the first and second friction reducing members may normally be adjacent to and spaced from each other. The hinge may form an opening sized and shaped for receiving the medical tubing. The opening, the first friction reducing member, and the second friction reducing member may be aligned generally along the longitudinal axis. The opening may also be configured to orient the apparatus such that the hinge is perpendicular to the medical tube.

The first friction reducing member may include a first roller that is configured to rotate as the body portion slides along the length of the flexible tube. Similarly, the second friction reducing member may include a second roller that is configured to rotate as the body portion slides along the length of the flexible tube. The first and second friction reducing members may not normally be in contact, and the hinge may be biased to normally maintain the first and second friction reducing members in the spaced relationship. When the first and second friction reducing members are in contact, the hinge may be biased to urge the arms apart. The apparatus may normally allow fluid through the medical tube.

The apparatus may also include a medical tubing extending generally along the longitudinal axis, passing through the opening, and contacting the first and second friction reducing members. The apparatus may also include a third friction reducing member that includes a third roller. The third roller may be configured to rotate as the body portion slides along the length of the flexible/medical tube, and the flexible/medical tube may contact the third friction reducing member.

In some embodiments, the body portion may include a clip that secures the apparatus to the medical tube. The clip may extend from the first arm to the second arm, and a portion of the clip may engage the second arm of the body. Additionally or alternatively, the body may include a protrusion extending from an inner surface of the second arm. The protrusion may press the flexible tube against an opposing surface of the body (e.g., the first arm) to secure the apparatus to the medical tube.

The first arm may also include a first support member and a second support member. The first support member may be configured to receive a first end of the first friction reducing member. The second support member may be configured to receive a second end of the first friction reducing member. The first and second support member may secure the second friction reducing member to the body.

Additionally, the first support member may include a first recess for receiving the first end to secure the first friction reducing member to the first support member. The first recess may also be configured to allow the first friction reducing member to rotate and be removed from the first support member. The second support member may include a second recess for receiving the second end to secure the first friction reducing member to the second support member. The second recess may also be configured to allow the first friction reducing member to rotate and be removed from the second support member.

In accordance with additional embodiments, a system for draining a wound site may include an inlet member insertable into the wound site, a reservoir bulb for storing fluids drained from the wound site, a flexible tube extending from the inlet member to the reservoir bulb, and a tube clearing device. The flexible tube may fluidly connect the inlet member and the reservoir bulb.

The tube clearing device may be configured to clear fluid from the flexible tube, and may include a first arm having a first friction reducing member, a second arm having a second friction reducing member, and a hinge. The hinge (e.g., a living hinge) may connect the first and second arms to form a body that a has a generally longitudinal axis. The first arm may be moveable relative to the second arm, and the first and second friction reducing members may normally be adjacent to and spaced from each other. The hinge may form an opening sized and shaped for receiving the medical tubing. The opening, the first friction reducing member, and the second friction reducing member may be aligned generally along the longitudinal axis. The opening may also be configured to orient the apparatus such that the hinge is perpendicular to the medical tube.

The first friction reducing member may include a first roller that is configured to rotate as the body portion slides along the length of the flexible tube. The second friction reducing member may include a second roller that is configured to rotate as the body portion slides along the length of the flexible tube. The first and second friction reducing members may not normally be in contact, and the hinge may be biased to normally maintain the first and second friction reducing members in the spaced relationship. When the first and second friction reducing members are in contact, the hinge may be biased to urge the arms apart. The apparatus may normally allow fluid through the medical tube.

The medical tubing may extend generally along the longitudinal axis, pass through the opening, and contact the first and second friction reducing members. The apparatus may also include a third friction reducing member located on the first or second arm. The third friction reducing member may include a third roller.

The body may also include a clip that secures the apparatus to the flexible tube. The clip may extend from the first arm to the second arm, and a portion of the clip may engage the second arm of the body. Additionally or alternatively, the body may include a protrusion extending from an inner surface of the second arm. The protrusion may press the flexible tube against an opposing surface of the first arm to secure the apparatus to the flexible tube.

The body portion may also include first and second support members configured to receive a portion (e.g., an end) of a friction reducing member and secure the friction reducing member to the body. The first and second support members may include recesses for receiving the portion of the friction reducing member(s) to secure the first friction reducing member to the support members and body portion. The recesses may also be configured to allow the friction reducing member(s) to rotate and be removed from the support members.

In further embodiments, a method for clearing a tube within a wound drain may include (1) providing a wound drain having an inlet member, a reservoir, and a tube, and (2) placing a tube clearing device onto the tube. The inlet member may be insertable into the wound site, and the reservoir bulb may receive/collect fluid draining from the wound site. The flexible tube may extend between and fluidly connect the inlet member and reservoir bulb. The tube clearing device may include a first arm having a first friction reducing member, a second arm having a second friction reducing member, and a hinge. The hinge (e.g., a living hinge) may connect the first and second arms to form a body that a has a generally longitudinal axis. The first arm may be moveable relative to the second arm, and the first and second friction reducing members may normally be adjacent to and spaced from each other.

The hinge may form an opening sized and shaped for receiving the medical tubing. The opening, the first friction reducing member, and the second friction reducing member may be aligned generally along the longitudinal axis. The opening may also be configured to orient the apparatus such that the hinge is perpendicular to the medical tube. The method may also include (1) applying a force to the body portion to flex the hinge and cause the first and second arms to move towards one another (e.g., to compress/close the tube), and (2) sliding (e.g., from the inlet member to the reservoir bulb) the tube clearing device along the length of the tube to force fluid within the tube into the reservoir bulb.

In accordance with still further embodiments, a system for draining a wound site may include an inlet member insertable into the wound site, a reservoir bulb for storing fluids draining from the wound site, a flexible tube extending from and fluidly connecting the inlet member and the reservoir, and a tube clearing device. The tube clearing device may be configured to clear fluid from the flexible tube, and may include a first arm having a first friction reducing member, a second arm having a second friction reducing member, and a living hinge connecting the first and second arms to form a body. The first arm may be moveable relative to the second arm, and the first and second friction reducing members may normally be adjacent to and spaced from each other.

The body may have a generally longitudinal axis, and the first and second friction reducing members may be generally aligned along and parallel to the longitudinal axis. The first and second friction members may be spaced from one another such that the tube clearing device is configured to receive the flexible tube (e.g., medical tubing) between the first and second friction reducing members to secure the tube clearing device to the medical tube. The tube clearing device may allow fluid flow through the flexible tube when placed on the flexible tube.

The first friction reducing member may include a first roller, and the second friction reducing member may include a second roller. The body portion may also include a third friction reducing member located on the first arm (or second arm) of the body. The third friction reducing member may include a third roller. The first and second friction reducing members may normally not be in contact and the hinge may be biased to normally maintain the first and second friction reducing members in the spaced relationship. When the first and second friction reducing members are in contact, the hinge may be biased to urge the arms apart.

The body portion may also include a clip that secures the tube clearing device to the flexible tube. The clip may extend between the first arm and the second arm. A portion of the clip may engage second arm. Additionally or alternatively, the body may include a protrusion extending from an inner surface of the second arm. The protrusion may press the flexible tube against an opposing surface of the first arm to secure the tube clearing device to the flexible tube.

The body may also include first and second support members that are configured to receive the ends of the friction reducing members and secure the friction reducing members to the body. Each of the support members may include a recess for receiving the respective portions of the friction reducing members. The recesses may allow the friction reducing member(s) to rotate and be removed from the support member(s).

In accordance with additional embodiments of the present invention, a method for clearing a tube within a wound drain may include providing a wound drain and placing a tube clearing device onto a flexible tube within the wound drain. The wound drain may include an inlet member insertable into the wound site, a reservoir bulb for receiving fluid draining from the wound site, and the flexible tube that extends between and fluidly connects the inlet member and reservoir bulb. The tube clearing device may be configured to clear fluid from the flexible tube, and may include a first arm having a first friction reducing member, a second arm having a second friction reducing member, and a living hinge connecting the first and second arms to form a body. The first arm may be moveable relative to the second arm, and the first and second friction reducing members may normally be adjacent to and spaced from each other.

The body may have a generally longitudinal axis, and the first and second friction reducing member may be generally aligned along and parallel to the longitudinal axis. The first and second friction members may be spaced from one another such that the tube clearing device is configured to receive the flexible tube (e.g., medical tubing) between the first and second friction reducing members to secure the tube clearing device to the medical tube. The tube clearing device may allow fluid flow through the flexible tube when placed on the flexible tube.

The method may also include (1) applying a force to the body to flex the hinge and cause the first and second arm to move towards one another, and (2) sliding the tube clearing device along the length of the tube to force fluid within the tube into the reservoir bulb. When sliding the tube clearing device along the length of the tube, the method may slide the device from the inlet member to the reservoir bulb. The first and second friction reducing members may compress the flexible tube as the first and second arms move towards one another.

In accordance with further embodiments, a system for draining a wound site may include (1) an inlet member insertable into the wound site, (2) a reservoir bulb for storing fluids draining from the wound site, (3) a medical tube extending from and fluidly connecting the inlet member to the reservoir, and (4) a sleeve located over at least a portion of the flexible tube. The sleeve may have a low-coefficient of friction inner surface that is configured to contact an outer surface of the portion of the flexible tube. The sleeve may be deformable and may be configured to slide along a length of the flexible tube.

The sleeve may be a tube having an inner diameter that is larger than an outer diameter of the medical tube, and may be placed around the medical tube prior to assembly of the inlet member, reservoir bulb, and flexible tube (e.g., prior to assembly of the wound drain). The sleeve may be made from a low-coefficient of friction material and/or from an oil-impregnated substrate. In some embodiments, the inner surface of the sleeve may have at least one friction reducing member. For example, the at least one friction reducing member may include one or more ribs that reduce the surface to surface contact between the sleeve and the flexible tube.

In accordance with additional embodiments, an apparatus for clearing fluid from medical tubing may include a first arm, a second arm, a first spring hinge, and a second spring hinge. The first arm may have a first friction reducing member, and the second arm may have a second friction reducing member. The first and second spring hinge may connect the first and second arms to form a body which may, in turn, have a generally longitudinal axis. The first and second spring hinges may be on opposing sides of the body.

The first arm may be moveable relative to the second arm, and the first and second friction reducing members normally may be adjacent to and spaced from each other. Also, the first friction reducing member and second friction reducing member may be aligned generally along the longitudinal axis. Each of the friction reducing members may have a roller. The first and second friction reducing members may not normally be in contact, and the first and second spring hinges may be biased to normally maintain the first and second friction reducing members in the spaced relationship. When the first and second friction reducing members are in contact, the first and second spring hinges may be biased to urge the arms apart.

The apparatus may also include a medical tubing extending generally along the longitudinal axis. The medical tubing may pass between the first and second spring hinges and contact the first and second friction reducing members. The body may include a clip that secures the apparatus to the medical tubing. For example, the clip may extend from the first arm to the second arm, and at least a portion of the clip may engage the second arm. Additionally or alternatively, the body may include a protrusion extending from an inner surface of the second arm. The protrusion may press the medical tube against an inner surface of the first arm to secure the body to the medical tube.

The apparatus may normally allow fluid flow through the medical tube when placed on the medical tube. The apparatus may also include a third friction reducing member on the first arm (or the second arm). The third friction reducing member may include a roller. The first arm may include a first support member configured to receive a first end of the first friction reducing member, and a second support member configured to receive a second end of the first friction reducing member. The first and second support members may secure the first friction reducing member to the body.

The first support member may include a first recess and the second support member may include a second recess. The first and second recess may receive the ends (a first and second end, respectively) of the first friction reducing member. The first and second recesses may also be configured to allow the first friction reducing member to rotate and be removed from the support members. The first and second spring hinges may be integral with the body.

### BRIEF DESCRIPTION OF THE DRAWINGS

The foregoing features of embodiments will be more readily understood by reference to the following detailed description, taken with reference to the accompanying drawings, in which:

FIG. 1 schematically shows a prior art wound drain that may be used with embodiments of the present invention.

FIGS. 2A-2D schematically show an apparatus for clearing fluid from medical/drain tubing, in accordance with various embodiments of the present invention.

FIGS. 3A-3B schematically show an alternative embodiment of the apparatus shown in FIGS. 2A-2D placed on and compressing the medical tubing, in accordance with some embodiments of the present invention.

FIGS. 4A-4B schematically show alternative embodiments of the apparatus for clearing the medical tubing having more than two friction reducing members, in accordance with additional embodiments of the present invention.

FIGS. 5A-5B schematically show additional alternative embodiments of an apparatus for clearing fluid from medical tubing in accordance with various embodiments of the present invention.

FIGS. 6A-6D schematically show a further alternative embodiment of an apparatus for clearing fluid from medical tubing in accordance with various embodiments of the present invention.

FIGS. 7A-7E schematically show yet another alternative embodiment of an apparatus for clearing fluid from medical tubing in accordance with various embodiments of the present invention.

FIGS. 8A-8B schematically show a further alternative embodiment of an apparatus for clearing fluid from medical tubing in accordance with various embodiments of the present invention.

FIGS. 9A-9B schematically show various embodiments of the present invention packaged with a wound drain, in accordance with further embodiments of the present invention.

### DETAILED DESCRIPTION OF SPECIFIC EMBODIMENTS

Illustrative embodiments of the medical tube clearing device of the present invention have numerous advantages over the prior art and ameliorate the disadvantages of the prior art described above. These advantages will be apparent from the description as set forth below.

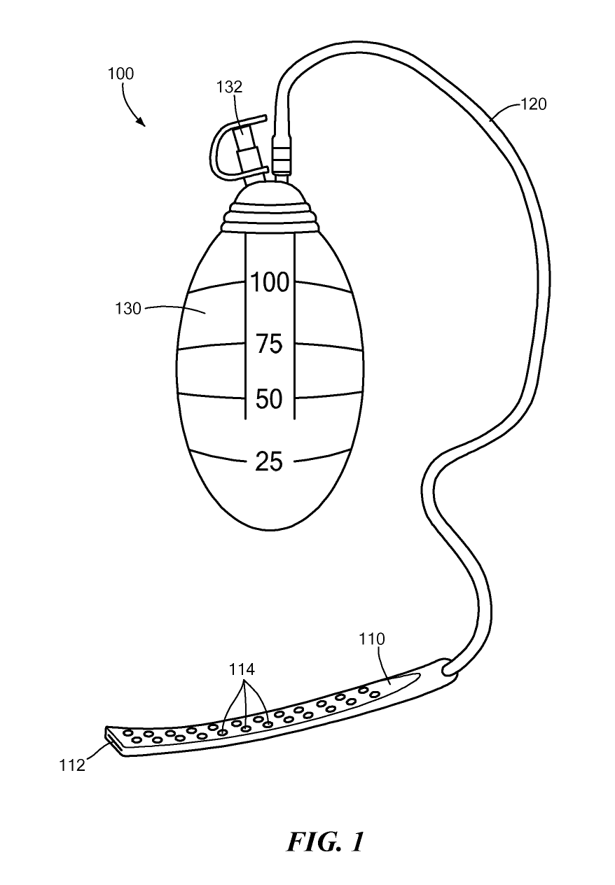

As discussed above, wound drains are used to capture excess bodily fluids during patient recovery. As shown in FIG. 1, a typical wound drain includes an inlet 110, a reservoir bulb 130, and a flexible tube 120 attached to the inlet 110 and fluidly connecting the inlet 110 and the bulb 130. The inlet 110 includes a flexible chamber 112 with perforated holes 114, and may be placed subcutaneously in the patient after a surgical procedure (e.g., into a wound site). Once inserted into the patient, fluid from the wound site may drain into the inlet 110 and flexible chamber 112 (e.g., thought the perforated holes 114), through the tube 120 and into the reservoir bulb 130. When the reservoir bulb 130 becomes full, the patient, a nurse or other individual may drain the bulb 130 using a drain port 132 located on the bulb 130.

In addition to draining the bulb 130, the tube 120 must also be drained. Various embodiments of the present invention simplify the “milking” and “stripping” process described above and reduce the physical dexterity required to drain the tube 120. Additionally, various embodiments also reduce patient discomfort by reducing the forces (e.g., the tugging) on the wound site.

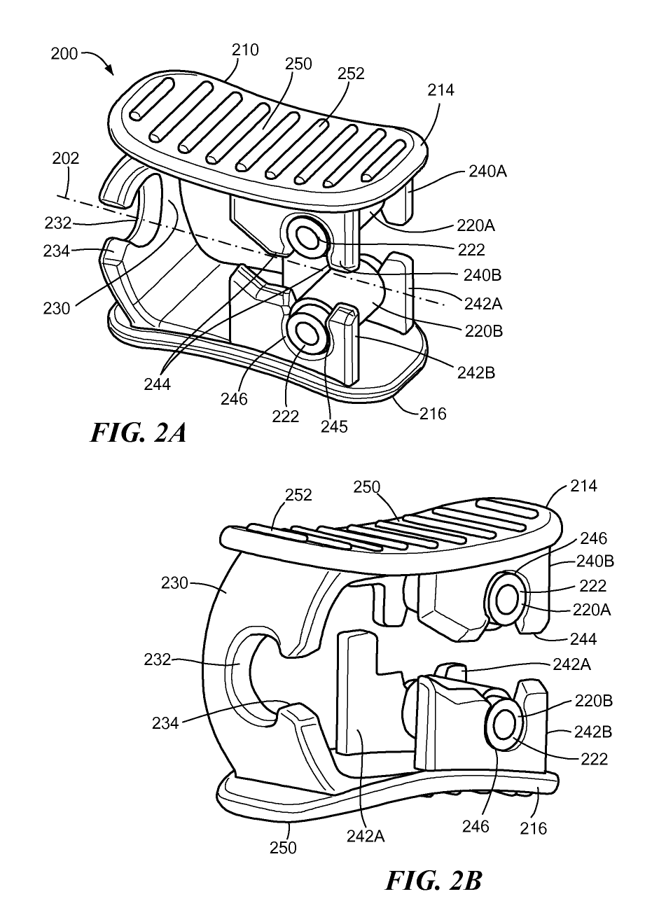

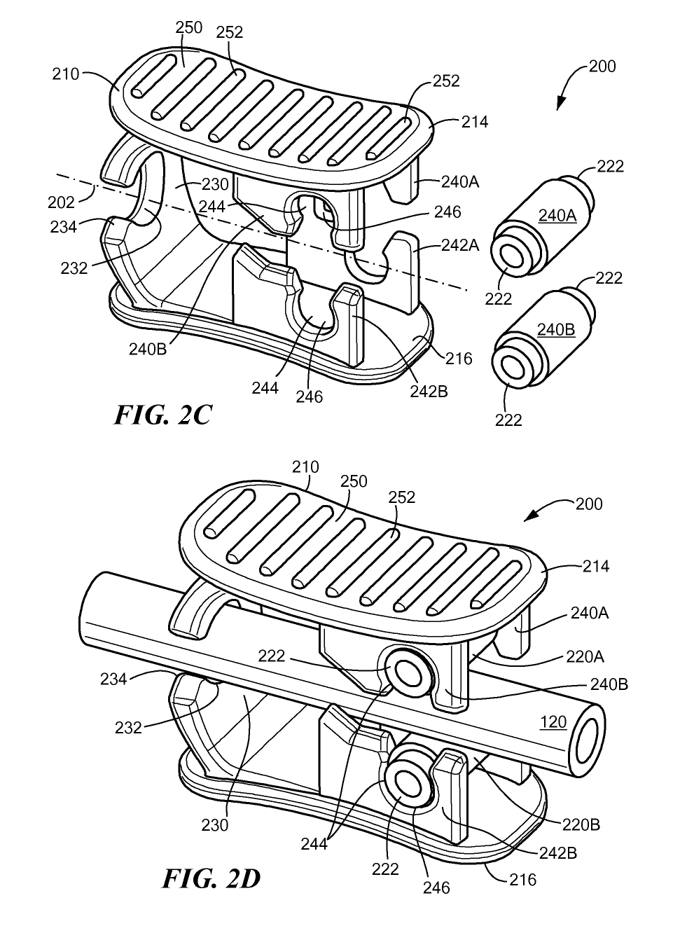

FIGS. 2A-2D schematically show perspective views of a first embodiment of a tube clearing device 200. In illustrative embodiments, the tube clearing device 200 has a body 210 defining the structure of the device and a number (e.g., two or more) friction reducing members 220 (e.g., rollers). Additionally, the body 210 may include a hinge 230 (e.g., a living hinge integrally formed within the body portion 210) that allows the device 200 to flex and connects a first arm 214 of the body 210 (e.g., the top of the body 210) with a second arm 216 of the body 210 (e.g., the bottom of the body 210). For example, the living hinge 230 may allow the first arm 214 and second arm 216 of the body portion 210 to flex towards one another in order to close the friction reducing members 220 on the tube 120 (discussed in greater detail below).

As shown in FIGS. 2A-2D, the friction reducing members 220 are not normally in contact with one another (e.g., they are spaced apart from one another). To that end, the hinge 230 may be biased in such a manner so as to keep the friction reducing members 220 in the spaced configuration (e.g., the hinge 230 may bias the arms 214/216 and friction reducing members 220 away from one another).

In order to secure/attach the friction reducing members 220 to the body portion 210, the device 200 may also include upper support members 240A/240B and lower support members 242A/242B. The upper support members 240A/240B secure the first friction reducing member 220A to the first arm 214 of the body portion 210, and the lower support members 242A/242B secure the second friction reducing member 220B to the second arm 216 of the body portion 210.

As discussed in greater detail below, the tube clearing device 200 may be placed over the tube 120 of a wound drain 100 and used to clear fluid from the tube 120. In order to allow various embodiments of the tube clearing device 200 to accommodate a variety of tube sizes, the friction reducing members 220A/220B may be removable from the support members 240A/240B/242A/242B. To that end, each of the support members 240A/240B/242A/242B may have an open end 244 that allows the ends 222 of the friction reducing members 220A/220B to “snap” into a hole 246 extending through the support members 240A/240B/242A/242B. For example, the width of the opening in the open end 244 may be smaller than the hole 246 such that the end 222 of the friction reducing member 220A/220B may be pressed into the larger diameter hole 246 which, in turn, allows the friction reducing members 220A/220B to rotate. In order aid in insertion, the opening in the open end 244 may have an angled surface 245.

As mentioned above, the tube clearing device 200 may be placed over/on the tube 120. For example, as shown in FIG. 2D, the tube clearing device 200 may be placed over the tube such that the hinge 230 is perpendicular to the tube 120 (or the longitudinal axis 202 of the device 200), and the tube 120 passes through an opening 232 extending through the hinge 230 and sized and shaped to receive the flexible tube 120 (e.g., the medical tubing). As best shown in FIG. 2A, the opening 232 within the hinge 230, and the first and second friction reducing members 220A/220B may be generally aligned along the longitudinal axis 202 of the tube clearing device.

In a manner similar to the hole 246 and open end 244 of the support members 240A/240B/242A/242B discussed above, one side of the hinge 230 may also be open (e.g., it may have a side opening 234) to allow the tube 120 to “snap” into place within the opening 232 (e.g., the tube 120 may deform slightly as it passes through the side opening 234 of the hinge 230 and return to its undeformed state once in place within the opening 232). The side opening allows the tube clearing device 200 to be placed on the tube 120 after assembly of the WOUND drain 100.

It is important to note that, when the tube clearing device 200 is placed on the tube 120 and the hinge 230 is not flexed (e.g., when the tube clearing device 200/body portion 210 is/are in the undeformed/at-rest/space apart configuration), the friction reducing members 220A/220B do not prevent flow through the tube. However, in order to prevent the tube clearing device 200 from sliding along the tube 120 and/or prevent tube 120 from sliding out from between the friction reducing members 220A/220B, the friction reducing members 220A/220B may apply a small force on the tube 120 to keep the tube clearing device 200 in place. This force, however, should not be large enough to significantly compress the tube 120 and restrict flow through the tube 120.

Some embodiments of the tube clearing device 200 may include finger tabs 250 located on the first arm 214 and second arm 216 of the body 210. The finger tabs 250 provide users with a place to grip/hold the tube clearing device 200 (e.g., with the thumb and index finger) when in use. Each of the finger tabs may include one or more ribs 252 to help improve grip and minimize the chance that the user's fingers will slip off of the clearing device 200.

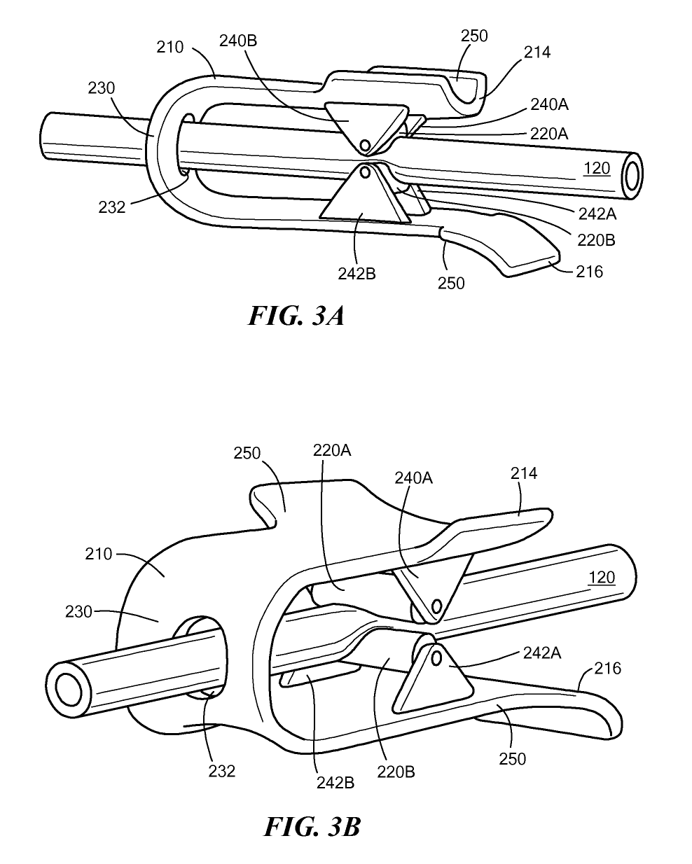

Once placed on the tube 120, the patient, nurse, or other user may use the tube clearing device 200 to drain the tube 120 and force any fluid, particulates, and/or occlusions into the reservoir bulb 130. To that end, the user may grasp the tube clearing device 200 between their thumb and index finger (e.g., on the finger tabs 250), and squeeze the tube clearing device 200. Squeezing the device 200 in this manner will cause the first arm 214 and second arm 216 of the body portion 210 to deform/flex towards one another (e.g., about the hinge 230), and close the friction reducing members 220A and 220B on the tube 120 (see FIGS. 3A and 3B). As the friction reducing members 220A/220B close on the tube 120, the tube 120 will deform and essentially close (e.g., fluid within the tube 120 will not be able to flow past the friction reducing members 220A/220B).

Once the friction reducing members 220A/220B close on the tube 120, the user may then move the tube clearing device 200 towards the reservoir 130 by sliding it down the length of the tube 120. As the user moves the tube clearing device 200 towards the reservoir 130, the friction reducing members 220A/220B (e.g., the rollers) will rotate and force any residual fluid/particulates/occlusions within the tube 120 towards and into the reservoir 130.

It should be understood that, in order to remove as much fluid as possible from the tube 120, it is preferable to place the tube clearing device 200 as close as possible to the wound site (e.g., as close as possible to where the tube 120 enters the wound site/patient). Also, in order minimize patient discomfort (e.g., caused by the tugging/pulling on the tube 120) and prevent the tube 120 from pulling out of the wound site, the user should grasp the tube 120 near the wound site as they move the tube clearing device 200 down the length of the tube 120.

Unlike the prior art methods of milking/stripping the tube 120, various embodiments of the present invention greatly reduce the amount of force required to clear the tube 120. In particular, the friction reducing members 220A/220B (e.g., the rollers) greatly reduce friction between the tube clearing device 200 and the tube 120 (e.g., as compared to the friction between the users fingers and the tube 120 in prior art methods) which, in turn, reduces the force required to clear the tube 120. By reducing the force required to clear the tube 120 (e.g., the force required to move the clearing device 200 along the length of the tube 120), the force required to prevent tugging/pulling on the wound site and prevent the drain tube 120 from being pulled out of the wound site is similarly reduced. Additionally, reducing the amount of force required to clear the tube 120 simplifies the clearing process and reduces the dexterity required to clear the tube 120.

Once the tube 120 is sufficiently cleared (e.g., all fluid/particulates/occlusions have been forced into the reservoir 130), the user may simply release the tube clearing device 200. As the user releases the tube clearing device 200, the hinge 230 will return to its at-rest state and cause the first arm 214 and second arm 216 of the body portion 210 to flex away from one another towards their spaced apart configuration (shown in FIG. 2D) This, in turn, re-opens the tube 120 so that fluid may, once again, flow through the tube 120 (e.g. from the wound site/inlet 110 to the reservoir 130).

It is important to note that, as shown in FIGS. 3A and 3B, some embodiments of the tube clearing device may not have the side opening 232 within the hinge 230. Rather, the hinge opening 232 may simply be a through-hole. In such embodiments, the tube clearing device 200 should be installed (e.g., slid over) on the tube 120 prior to assembly of the components of the wound drain 100. Similarly, the support members 240A/240B/242A/242B may also not have an open end 244. Rather, the support members 240A/240B/242A/242B may simply have the hole 246 extending through the members.

Although the friction reducing members 220A/220B in the above described embodiments include rollers, other embodiments of the present invention may have different friction reducing members. For example, some embodiments may have one or more thin anvil like structures (e.g., thin projections extending from the first and second arms 214/16). Unlike the friction reducing members shown in FIGS. 2A-2D, these anvil like structures may not have rollers and may not be moveable (e.g., they are essentially stationary). In such embodiments, the end of the structure may contact the tube 120 and the reduction in friction may be caused by the relatively small contact area between the end of the structure (e.g., because the structure is thin) and the tube 120.

Some embodiments of the present invention may have a combination of non-moveable friction reducing members (e.g., the thin anvil like structures discussed above) and moveable friction reducing members (e.g., the rollers). For example, the tube clearing device 200 may have a roller 220A on the first arm 214 of the body 210 and the thin structure on the second arm 216. Alternatively, the tube clearing device may have a roller 220B on the second arm 216 of the body 210 and the thin structure on the first arm 214. Other embodiments may not have any rollers and may just have the thin anvil structures on both the first arm 214 and second arm 216.

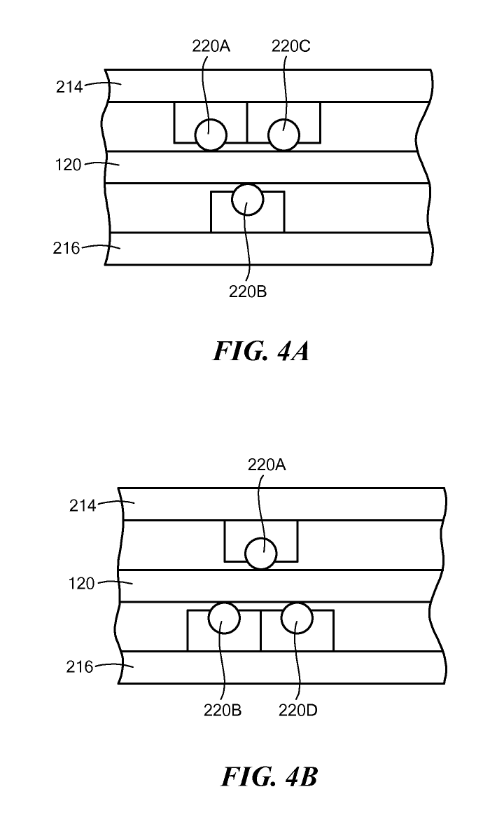

Additionally or alternatively, some embodiments may have more than two friction reducing members 220A/220B. For example, as schematically shown in FIGS. 4A and 4B, the tube clearing device 200 may have three or more friction reducing members. In such embodiments, the device 200 may have two friction reducing members 220A/220C on the first arm 214 of the body 210 and one friction reducing member 220B on the second arm 216 (FIG. 4A). Alternatively, the device 200 may have two have two friction reducing members 220B/220D on the second arm 216 of the body 210 and one friction reducing member 220A on the first arm 214 (FIG. 4B).

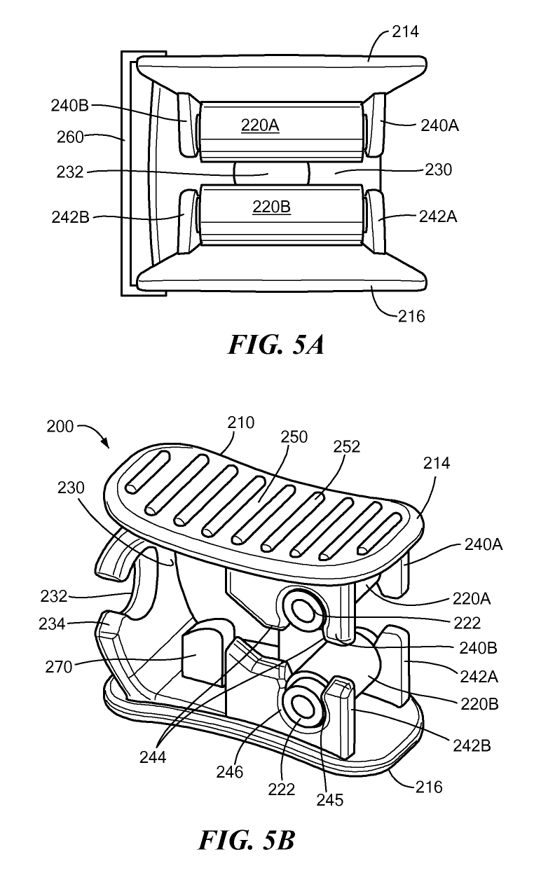

In order to help secure the tube clearing device 200 to the drain tube 120, some embodiments may have a clip or clamp-like structure. For example, as shown in FIG. 5A, the body portion 210 may include a clip 260 extending between the first arm 214 and second arm 216 of the body 210. Once the device 200 is placed on the drain tube, the user may engage the clip 260 with the second arm 216 of the body 210. Once engaged, the clip 260 will provide a light clamping force (e.g., by slightly deforming the hinge 230) to secure the device 200 to the drain tube (without pinching the tube 120 closed).

Additionally or alternatively, the device 200 may have a flexible protrusion 270 extending from either the first arm 214 or second arm 214 of the body 210 (FIG. 5B shows the protrusion 270 extending from the second arm 214). In such embodiments, the protrusion 270 may press against one side of the drain tube 120 and force the drain tube 120 against the opposing side of the device 200 (e.g., against the first arm 214 in FIG. 5B). The force applied by the protrusion 270 may be large enough to attach/secure the device 200 on the drain tube 200 without pinching the tube 120 closed or hindering movement of the device 200 along the length of the tube 120 during operation (e.g., during tube clearing).

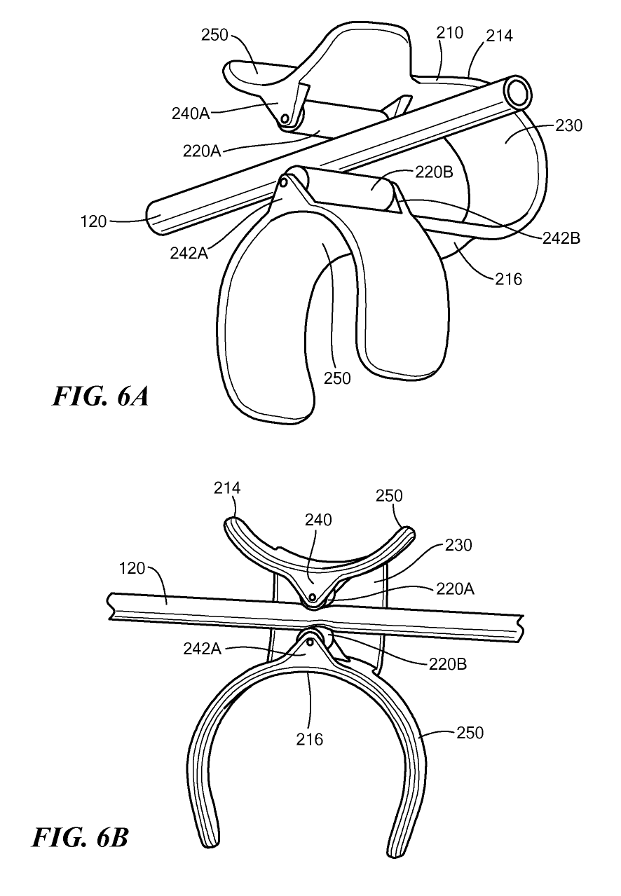

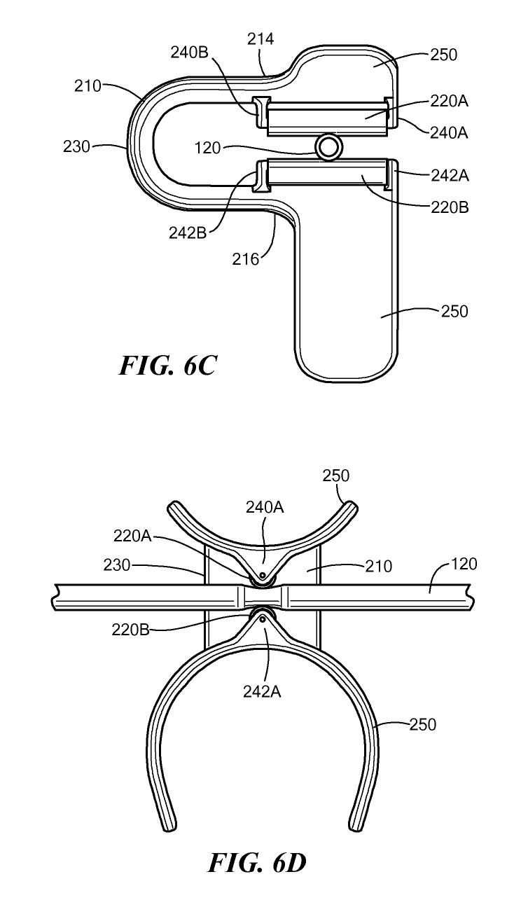

Although the above described embodiments describe a tube clearing device 200 with a hinge 230 that is perpendicular to the tube 120, other embodiments of the present invention may have hinges 230 with different orientations. For example, the hinge 230 may be oriented at another angle with respect to the tube 120 (e.g., at a 45 degree angle with respect to the tube 120). Alternatively, as shown in FIGS. 6A-6D, the hinge 230 may be located parallel to the tube 120 when the device 200 is placed on the tube 120. In such embodiments, operation of the tube clearing device 200 is similar to that of the embodiments shown in FIGS. 2A through 2D. Namely, once placed on the tube 120, the user may grasp the device 200 on the finger tabs 250 and squeeze the device 200 to flex the hinge 230 and close the friction reducing members 220A/220B on the tube 120. The user may then slide the device 200 along the length of the tube 120 to force the fluid in the tube 120 into the reservoir 130.

In a manner similar to the perpendicular hinge embodiment discussed above, the hinge 230 in the parallel embodiment may also be configured to provide sufficient force on the friction reducing members 220A/220B to prevent the device 200 from sliding along the tube 120 and/or falling off the tube 120, but not enough force to close the tube 120 or otherwise restrict the flow through the tube 120. For example, the hinge 230 may apply enough force that the friction reducing members 220A/220B contact the tube 120 and hold the tube 120 in place, but not enough force to significantly deform the tube 120.

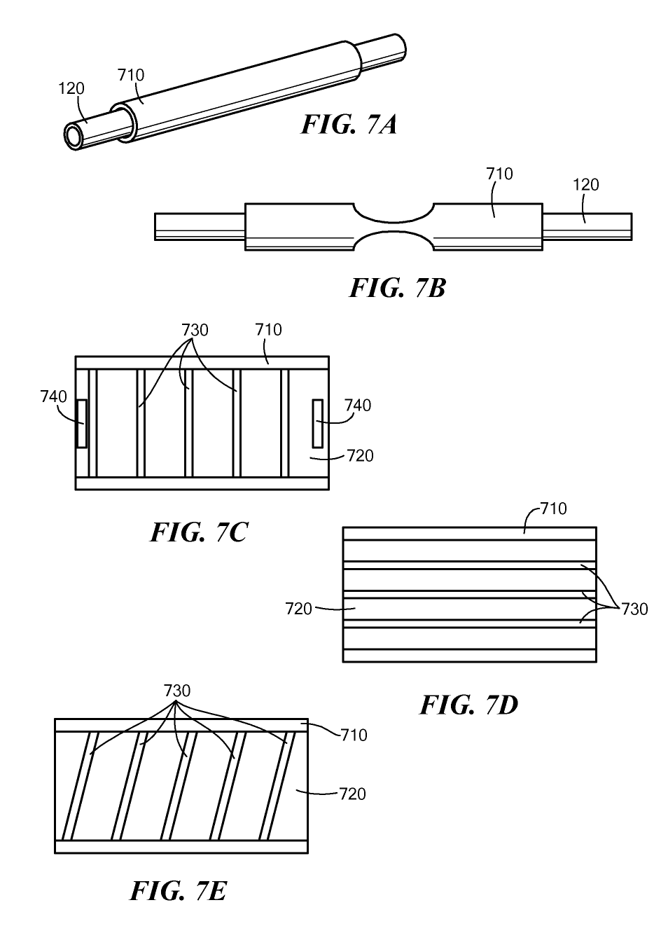

FIGS. 7A and 7B schematically show an additional embodiment of the present invention. In particular, some embodiments of the tube clearing device may consist of a friction reducing sleeve 710 that may placed over the drain tube 120. The friction reducing sleeve 710 may have an inner diameter that is slightly larger than the outer diameter of the drain tube 120 such that the friction reducing sleeve 710 may slide along the length of the drain tube 120. Additionally, in order to reduce the friction between the inner surface 720 (FIGS. 7C-7E) of the friction reducing sleeve 710 and the outer surface 122 of the drain tube 120, the inner surface 720 may have a low-coefficient of friction. To that end, the sleeve 710 may be made from a low-coefficient of friction material (e.g., PTFE) and/or the sleeve 710 may be made from an oil-impregnated substrate. The sleeve 710 may also have pads 740 (FIG. 7C) that hold a low coefficient of friction oil. In such embodiments, the pads 740 may release the oil as the sleeve 710 slides along the length of the tube 120 to further reduce friction.

In operation, when a user wishes to drain the drain tube 120, the user may squeeze the sleeve 710 such that the sleeve 710 deforms and compresses the drain tube 120, FIG. 7B. The user may then slide the sleeve 710 along the length of the drain tube 120 towards the reservoir 130 to force the fluid/particulates/occlusions within the drain tube 120 into the reservoir 130. In a manner similar to the rollers/friction reducing members 220A/220B discussed above, the low-coefficient of friction inner surface 720 (or oil released from the pads 740) reduces the force required to drain the drain tube 120.

In order to further reduce friction, some embodiments of the sleeve 710 may include a number of ribs 730 or similar structures on the inner surface 720. These structures (e.g., the ribs) reduce the surface contact between the sleeve 710 and the drain tube 120 and, therefore, further reduce the friction between them. As shown in FIGS. 7C-7E (showing cross-sections of the sleeve 710), the ribs 730 can be oriented perpendicular to the longitudinal axis of the drain tube 120 (FIG. 7C), parallel to the longitudinal axis of the drain tube 120 (FIG. 7D), or at an angle relative to the longitudinal axis of the drain tube 120 (FIG. 7E).

It is important to note that some embodiments of the sleeve 710 do not need to completely surround the drain tube 120. For example, the cross-section of the sleeve 710 may be “U” or “C” shaped and only surround a portion of the drain tube 120 (e.g., the sleeve 710 may only cover 180 degrees or 270 degrees of the circumference of the drain tube 120). In such embodiments, the sleeve 710 does not need to be installed over the drain tube 120 prior to assembly of the WOUND drain. Rather, the sleeve 710 may be placed over the drain tube 120 after assembly (e.g., just prior to the tube clearing process). Additionally, in embodiments in which the sleeve 710 surrounds greater than 180 degrees of the circumference of the drain tube 120 (e.g., sleeves that are “C” shaped), the sleeve 710 may remain on the drain tube 120 when not in use (e.g., because the sleeve material extending past 180 degrees prevents the sleeve 710 from falling off the tube 120).

The sleeve may also be used in conjunction with the tube clearing devices 200 discussed above. In particular, the tube clearing device 200 may be placed over a sleeve 710 on the tube 120. In such embodiments, when the tube clearing device 200 is squeezed, the friction reducing members 220A/220B will close on and deform the sleeve 710 which will, in turn, close on and deform the tube 120. The user may then slide the tube clearing device 200 and the sleeve 710 along the length of the tube 120.

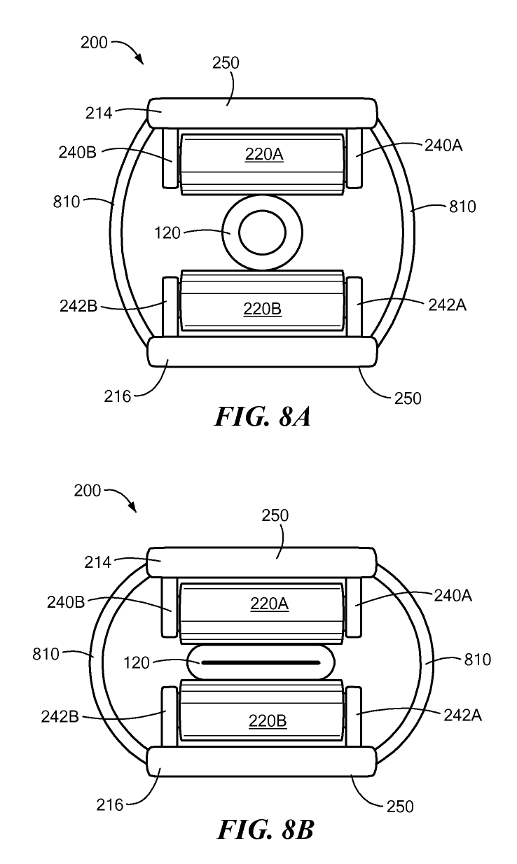

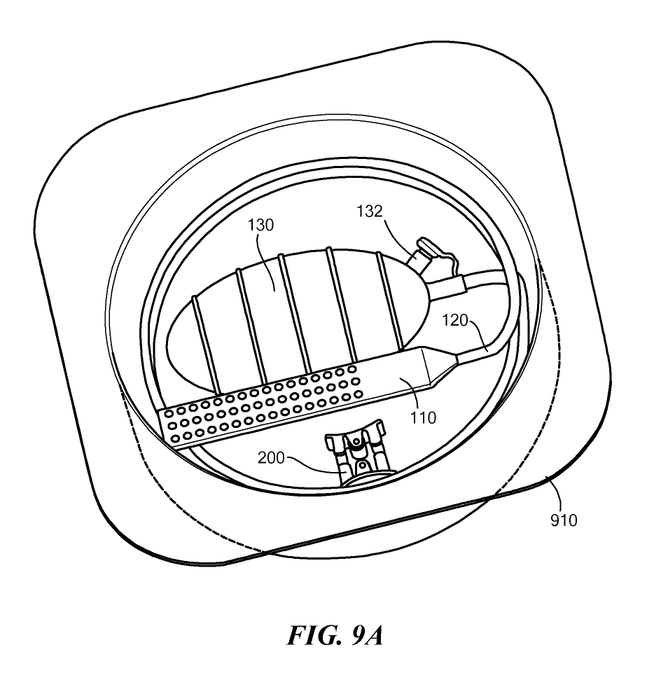

FIGS. 8A and 8B show a further embodiment of a tube clearing device. In particular, instead of having the hinge 230 described above, some embodiments may have one or more spring hinges 810 that connect(s) the first arm 214 and the second arm 216 of the body 210. The spring hinges 810 may be located on either side of the body 210 and, like the hinge 230 discussed above, deform as the user squeezes the device. For example, as the use squeezes the tube clearing device 200, the spring hinges 810 may deform/compress causing the first arm 214 and the second arm 216 to move towards one another which, in turn, causes the friction reducing members 220A/220B to close on the tube 120. As the friction reducing members 220A/220B close on the tube 120, the tube 120 will, in turn, collapse/close (as shown in FIG. 8B) to prevent flow past the tube clearing device 200. The user may then slide the tube clearing device 200 along the length of the tube 120 to force the fluid present in the tube 120 into the reservoir 130.

Once the user has completed the milking/stripping process and released the tube clearing device 200, the spring hinges 810, like hinge 230, will bias the first arm 214 and second arm 216 away from each other and allow the tube 120 to re-open (e.g., to return to the state shown in FIG. 8A). Once back in this configuration, fluid may once again flow from the wound site and through the tube 120.

It should be noted that, like the hinge 230 discussed above, the spring hinges 810 may be similarly configured to cause the friction reducing members 220A/220B to apply a small force on the tube 120 when the spring hinges 810 are not flexed/compressed (e.g., as shown in FIG. 8A). By applying this small force on the tube 120, the spring hinges 810 and friction reducing members 220A/220B prevent the tube clearing device 200 from sliding along the tube 120 and/or prevent the tube 120 from sliding out from between the friction reducing members 220A/220B when not in use.

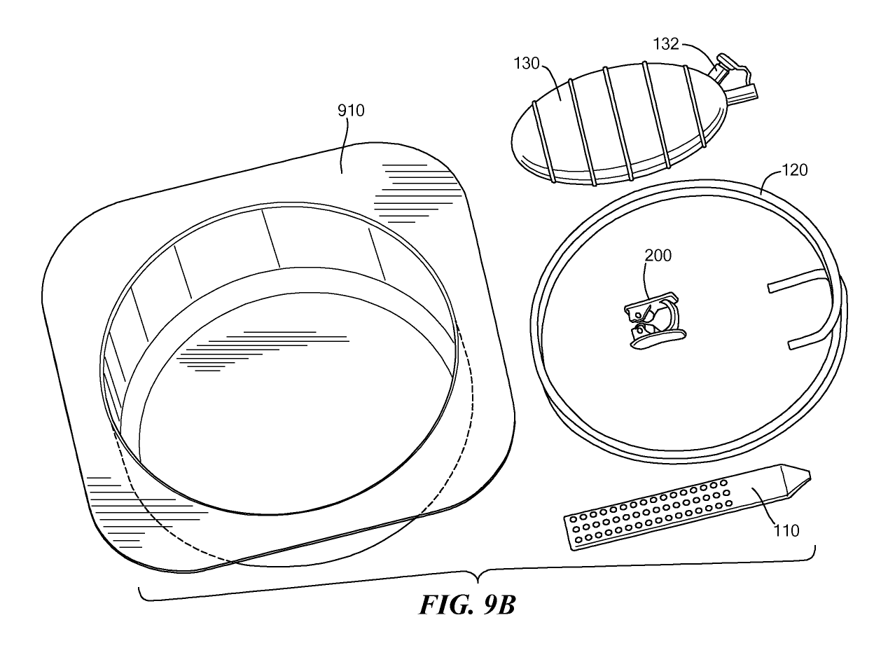

As shown in FIGS. 9A and 9B, the tube clearing device 200 may be pre-packaged with the components of the wound drain. For example, the reservoir 130 (e.g., reservoir bulb), drain tube 120, and the inlet 110 may be packaged together with the tube clearing device 200 (or sleeve 710 or both) in sterile packaging 910. The wound drain 100 may be packaged pre-assembled without the clearing device 200 installed (FIG. 9A) or with the clearing device 200 installed. Alternatively, the wound drain 100 may be packaged unassembled without the clearing device 200 installed (FIG. 9B) or with the clearing device 200 installed on the drain tube 120.

The embodiments of the invention described above are intended to be merely exemplary; numerous variations and modifications will be apparent to those skilled in the art. All such variations and modifications are intended to be within the scope of the present invention as defined in any appended claims.

What is claimed is:

1. An apparatus for clearing fluid from medical tubing, the apparatus comprising:
a first arm having a first friction reducing member;
a second arm having a second friction reducing member; and
a hinge connecting the first and second arms to form a body, the body having a generally longitudinal axis,
the first arm being movable relative to the second arm,
the first and second friction reducing members normally being adjacent to and spaced from each other,
the hinge forming an opening sized and shaped for receiving the medical tubing, the opening, first friction reducing member, and second friction reducing member being aligned generally along the longitudinal axis.

2. The apparatus as defined by claim 1 wherein the first friction reducing member comprises a roller.

3. The apparatus as defined by claim 2 wherein the second friction reducing member comprises a second roller.

4. The apparatus as defined by claim 1 wherein the first and second friction reducing members normally are not in contact, the hinge biased to normally maintain the first and second friction reducing members in the spaced relationship.

5. The apparatus as defined by claim 1 wherein when the first and second friction reducing members are in contact, the hinge is biased to urge the arms apart.

6. The apparatus as defined by claim 1 further comprising a medical tubing extending generally along the longitudinal axis, the medical tubing passing through the opening and contacting the first and second friction reducing members.

7. The apparatus as defined by claim 6, wherein the body includes a clip that secures the apparatus to the medical tubing.

8. The apparatus as defined by claim 7, wherein the clip extends from the first arm to the second arm, at least a portion of the clip engaging the second arm.

9. The apparatus as defined by claim 6, wherein the body includes a protrusion extending from an inner surface of the second arm, the protrusion pressing the medical tube against an inner surface of the first arm to secure the body to the medical tube.

10. The apparatus as defined by claim 1, wherein the opening is configured to orient the apparatus such that the hinge is perpendicular to the medical tube.

11. The apparatus as defined by claim 1, wherein the apparatus normally allows fluid flow through the medical tube when placed on the medical tube.

12. The apparatus as defined by claim 1, the apparatus further comprising a third friction reducing member located on the first arm.

13. The apparatus as defined by claim 12, wherein the third friction reducing member comprises a third roller.

14. The apparatus as defined by claim 1, wherein the first arm includes:
a first support member configured to receive a first end of the first friction reducing member; and
a second support member configured to receive a second end of the first friction reducing member, the first and second support members securing the first friction reducing member to the body.

15. The apparatus as defined by claim 14, wherein
the first support member includes a first recess for receiving the first end of the first friction reducing member, the first recess also configured to allow the first friction reducing member to rotate and be removed from the first support member, and
the second support member includes a second recess for receiving the second end of the first friction reducing member, the second recess also configured to allow the first friction reducing member to rotate and be removed from the second support member.

16. The apparatus as defined by claim 1, wherein the hinge is a living hinge.

17. A system for draining a wound site comprising:
an inlet member insertable into the wound site;
a reservoir bulb for storing fluids draining from the wound site;
a flexible tube extending from the inlet member to the reservoir bulb, the flexible tube fluidly connecting the inlet member and the reservoir bulb; and
a tube clearing device configured to clear fluid from the flexible tube, the tube clearing device comprising:
a first arm having a first friction reducing member;
a second arm having a second friction reducing member; and
a hinge connecting the first and second arms to form a body, the body having a generally longitudinal axis,
the first arm being movable relative to the second arm,
the first and second friction reducing members normally being adjacent to and spaced from each other,
the hinge forming an opening sized and shaped for receiving the flexible tube, the opening, first friction reducing member, and second friction reducing member being aligned generally along the longitudinal axis.

18-21. (canceled)

22. The system as defined by claim 17 wherein the flexible tube extends generally along the longitudinal axis, the flexible tube passing through the opening and contacting the first and second friction reducing members.

23. The system as defined by claim 22, wherein the body includes a clip that secures the apparatus to the flexible tube.

24. The system as defined by claim 23, wherein the clip extends from the first arm to the second arm, at least a portion of the clip engaging the second arm.

25-32. (canceled)

33. A method for clearing a tube within a wound drain comprising:
providing a wound drain, the wound drain including an inlet member insertable into a wound site, a reservoir bulb for receiving fluid draining from the wound site, and a flexible tube extending between and fluidly connecting the inlet member and reservoir bulb;
placing a tube clearing device onto the flexible tube, the tube clearing device comprising:
a first arm having a first friction reducing member;
a second arm having a second friction reducing member; and
a hinge connecting the first and second arms to form a body, the body having a generally longitudinal axis,
the first arm being movable relative to the second arm,
the first and second friction reducing members normally being adjacent to and spaced from each other,
the hinge forming an opening sized and shaped for receiving the flexible tube, the opening, first friction reducing member, and second friction reducing member being aligned generally along the longitudinal axis;
applying a force to the body to flex the hinge and cause the first and second arm to move towards one another, the first and second friction reducing members compressing the flexible tube as the first and second arm move towards one another; and
sliding the tube clearing device along the length of the tube to force fluid within the tube into the reservoir bulb.

34-100. (canceled)
